(19) United States
(12) Patent Application Publication
(10) Pub. No.: US 2008/0200741 A1
(43) Pub. Date: Aug. 21, 2008

(54) DEHYDROGENATION CATALYST, PROCESS FOR PREPARATION, AND A METHOD OF USE THEREOF

(76) Inventor: Ruth Mary KOWALESKI, Cypress, TX (US)

Correspondence Address: SHELL OIL COMPANY, P O BOX 2463, HOUSTON, TX, 772522463, US

(21) Appl. No.: 12/022,416

(22) Filed: Jan. 30, 2008

Related U.S. Application Data
(60) Provisional application No. 60/887,185, filed on Jan. 30, 2007.

(51) Int. Cl. C07C 2/64; B01J 23/745; B01J 23/83; B01J 23/78; B01J 23/888; B01J 23/88

(52) U.S. Cl. 585/444; 502/304; 502/316; 502/328; 502/338

(57) ABSTRACT

A process for preparing a dehydrogenation catalyst comprising preparing a mixture comprising a treated regenerator iron oxide and at least one additional catalyst component; and calcining the mixture wherein the treated regenerator iron oxide is prepared by washing a regenerator iron oxide at a temperature below 350° C. such that the treated regenerator iron oxide has a chloride content of at most 500 ppmw relative to the weight of iron oxide, calculated as Fe₂O₃; the catalyst prepared by this process and the use of the catalyst in a dehydrogenation process.

This application claims the benefit of U.S. Provisional Application No. 60/887,185, filed Jan. 30, 2007, which is hereby incorporated by reference.

### FIELD OF THE INVENTION

This invention relates to a dehydrogenation catalyst derived from a regenerator iron oxide, the preparation thereof and its use in a dehydrogenation process.

### BACKGROUND OF THE INVENTION

In the field of catalytic dehydrogenation of hydrocarbons there are ongoing efforts to develop improved catalysts that may be made at lower costs. One way of reducing the cost of iron oxide based dehydrogenation catalysts is to use lower cost raw materials. One such low cost raw material is regenerator iron oxide produced from hydrochloric acid waste liquid generated from steel pickling. The use of regenerator iron oxide as the iron oxide component of an iron oxide based dehydrogenation catalyst may provide significant cost savings in raw material costs in comparison to the use of other more conventional commercial iron oxides.

The use of regenerator iron oxide as a catalyst component is described in U.S. Pat. No. 5,401,485, which discloses catalysts containing iron oxide obtained by the spray roasting of a hydrochloric acid solution containing iron chloride. A significant disadvantage associated with the use of regenerator iron oxide as a catalyst component is that the resulting catalysts may have what is considered herein to be high levels of residual chloride that results in less effective dehydrogenation catalysts. The '485 patent discloses the use of sulfuric acid to reduce the residual chloride level in iron oxides.

The prior art also describes processes for providing a regenerator iron oxide that has a reduced chloride content, for example U.S. Patent Application Publication 2004/0097768 and U.S. Pat. No. 6,863,877. The processes described in both of these documents require a costly high temperature heat treatment step that has negative effects on the regenerator iron oxide being treated, such as reducing the surface area of the iron oxide.

Accordingly, it would be an advancement in the art to provide a dehydrogenation catalyst and process for forming the catalyst that uses low cost materials but provides a catalyst with a high initial activity.

### SUMMARY OF THE INVENTION

The invention provides a process for preparing a dehydrogenation catalyst comprising preparing a mixture comprising a treated regenerator iron oxide and at least one additional catalyst component; and calcining the mixture wherein the treated regenerator iron oxide is prepared by washing a regenerator iron oxide at a temperature below 350° C. such that the treated regenerator iron oxide has a chloride content of at most 500 ppmw relative to the weight of iron oxide, calculated as Fe₂O₃.

The invention provides a catalyst comprising treated regenerator iron oxide wherein the dehydrogenation catalyst comprises from 10 to 95 weight percent iron oxide relative to the total weight of the dehydrogenation catalyst, and calculated as Fe₂O₃, and from 5 to 40 weight percent potassium, calculated as K₂O.

The invention further provides a dehydrogenation process, comprising: providing a reactor loaded with a catalyst comprising treated regenerator iron oxide, introducing a feedstock into the reactor, and operating the reactor under conditions suitable to yield a dehydrogenation product.

The invention further provides a method of improving the operation of a dehydrogenation system, including a dehydrogenation reactor that is loaded with a non-regenerator iron oxide based dehydrogenation catalyst, and wherein said method comprises: removing from said dehydrogenation reactor said non-regenerator iron oxide based dehydrogenation catalyst; replacing the thus-removed non-regenerator iron oxide based dehydrogenation catalyst with a regenerator iron oxide based dehydrogenation catalyst comprising treated regenerator iron oxide to thereby provide a modified dehydrogenation system, including said dehydrogenation reactor that is loaded with said regenerator iron oxide based dehydrogenation catalyst; and operating said modified dehydrogenation system under dehydrogenation process conditions.

### DETAILED DESCRIPTION OF DRAWINGS

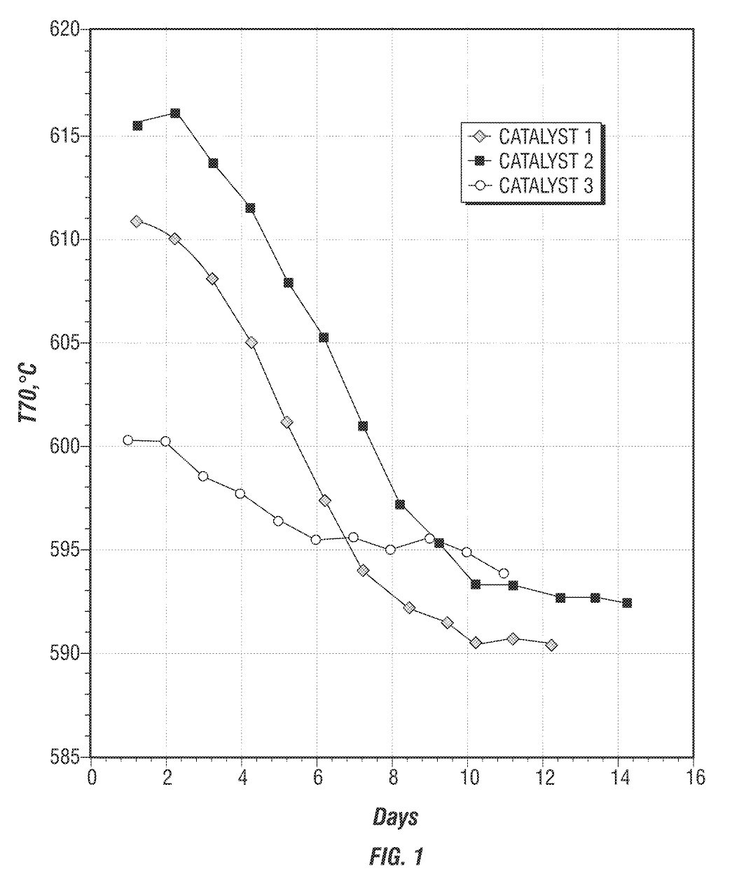

FIG. 1 presents comparative plots showing the calculated activity (T70) of tested catalysts as a function of time in days of use.

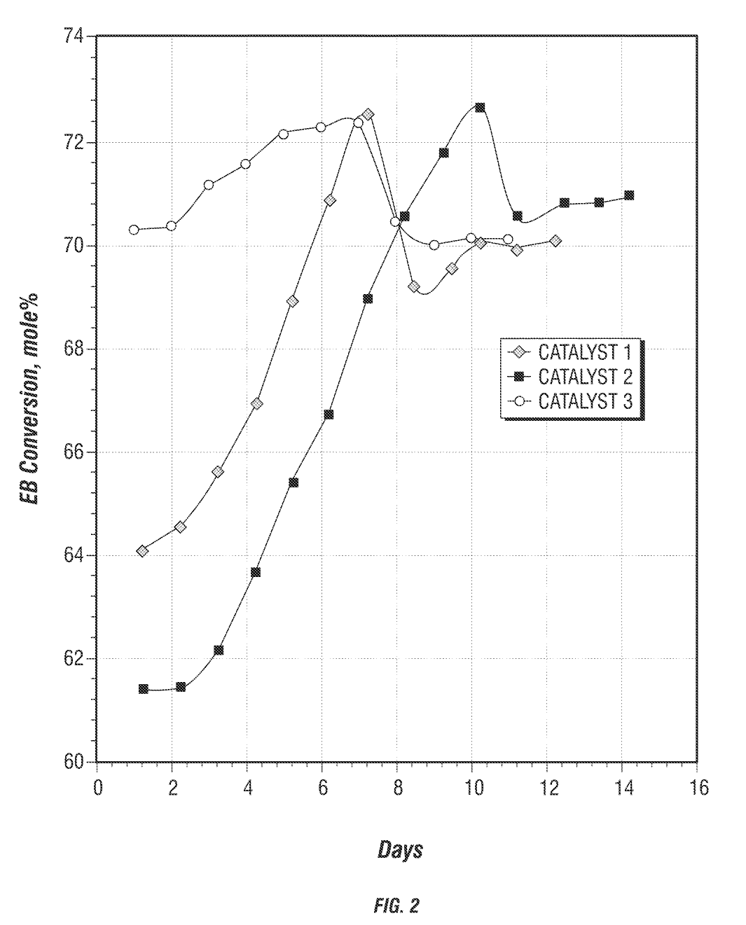

FIG. 2 presents comparative plots showing the actual conversion of ethylbenzene provided by tested catalysts as a function of time in days of use.

### DETAILED DESCRIPTION OF THE INVENTION

The present invention provides a catalyst that satisfies the need for lower cost iron oxide based catalysts. The use of a treating step at a temperature below 350° C. provides a regenerator iron oxide with a reduced chloride content and physical characteristics that are suitable for producing effective dehydrogenation catalysts. The surface area of the regenerator iron oxide provides more active sites for incorporation of additional catalyst components than other regenerator iron oxides that have been treated by high temperature heat treatments. The catalyst produced by the inventive method demonstrates a higher initial activity than other regenerator iron oxide based dehydrogenation catalysts that have not undergone a similar treating step to sufficiently reduce the residual chloride content.

The regenerator iron oxide component of the catalyst is an iron oxide derived from waste pickle liquor, an aqueous solution of hydrochloric acid and iron chloride waste liquid generated from steel pickling. This regenerator iron oxide, in general, may be made by the heat decomposition of iron chloride (either iron dichloride, or iron trichloride, or both) preferably in an oxidizing atmosphere, to yield iron oxide. More specifically, the regenerator iron oxide is made by spray roasting of waste pickle liquor by any method known to those skilled in the art, for example the Ruthner process described in U.S. Pat. No. 5,911,967.

The spray roasting may be conducted by spraying the waste pickle liquor into a roaster where it is exposed to a heated source of oxygen, for example air, and where the iron chloride, for example, FeCl₂ and/or FeCl₃, undergoes a thermal conversion to form iron oxide that is predominantly in the form of hematite (Fe₂O₃). The spray roasting may occur at spray roasting temperatures exceeding 300° C. and ranging upwardly to 800° C. or even higher temperatures. The iron oxide is recovered as regenerator iron oxide.

The regenerator iron oxide generally has an appreciable level of residual chloride content. The residual chloride content of such regenerator iron oxide is typically in the range of from about 700 ppmw to 20,000 ppmw, but more typically, the residual chloride concentration is in the range of from 800 ppmw to 10,000 ppmw. Thus the chloride concentration of the regenerator iron oxide may be greater than 1000 ppmw.

Methods of reducing the chloride concentration in the regenerator iron oxide have been discussed in the prior art, but each of these methods has disadvantages when the regenerator iron oxide is intended for use as a component in a dehydrogenation catalyst.

According to the present invention, the regenerator iron oxide is not calcined to remove chloride as taught by some of the prior art methods because this causes undesirable negative effects on the iron oxide structure. For example, calcining the regenerator iron oxide results in an iron oxide with a decreased surface area that can inhibit effective incorporation of additional catalyst components in the catalyst manufacturing process.

The regenerator iron oxide is treated by washing at a temperature below 350° C. to reduce the chloride content of the iron oxide. The treatment of the regenerator iron oxide may comprise acid washing, but preferably comprises water washing under suitable conditions to reduce the chloride content.

In one embodiment, the regenerator iron oxide is washed with an acid to reduce the chloride content. Acids suitable for use in the acid treatment of the regenerator iron oxide may include those acids that are decomposable to volatile compounds under the conditions of the catalyst preparation as described herein and can include organic acids and inorganic acids including those acids noted in U.S. Pat. No. 5,401,485 and U.S. Pat. No. 6,863,877, which are both incorporated herein by reference. Examples of organic acids include carboxylic acids such as formic acid, acetic acid and citric acid. Preferred organic acids include acetic acid and citric acid. Possible inorganic acids include carbonic acid, nitric acid, nitrous acid, phosphoric acid, phosphorous acid, sulfuric acid, and sulfurous acid. Among these acids, the preferred inorganic acids include nitric acid, nitrous acid, sulfuric acid, and sulfurous acid. The inorganic acids used in the acid treatment of the regenerator iron oxide are preferably in an aqueous solution having a Normality (equivalents of solute per liter of solution) in the range of from 0.01 to 4, preferably, from 0.05 to 3, and, most preferably, from 0.1 to 2.

In a preferred embodiment, the regenerator iron oxide is washed with water. The regenerator iron oxide may be simply washed with water to reduce the chloride content, but it is preferred to carry out certain steps prior to the water washing. It is preferred to prepare a slurry by contacting the regenerator iron oxide with water. The slurry may contain 5-25% solids by weight, although the percent of solids may be higher or lower. The addition of water to a regenerator iron oxide typically produces an acidic slurry, and the acidic nature of the slurry can make the remaining steps more difficult. It is preferred therefore to raise the pH of the slurry to make it easier to handle. The pH of the slurry is generally adjusted by adding a solution with a pH that is greater than the pH of the slurry solution. The solution that is added may be an aqueous solution comprising one or more of the following: sodium hydroxide, potassium hydroxide, calcium hydroxide, sodium carbonate, and lime. The slurry is then filtered, washed and dried. The resulting dried slurry material may undergo any number of filtering, washing and/or drying cycles. The resulting material may then be ground or deagglomerated after drying. Other embodiments of water washing are described in JP 5043252A, and Japanese Laid Open Patent Application No. 279045.

The pH adjustment of the slurry is optional, but generally results in a more efficient filtration step. The pH of the slurry is preferably increased to a pH of at least 5, and it may be increased even more, for example to a pH of 7. The pH however may be raised to any level above the pH of the original slurry.

The washing times, volumes and number of cycles can be varied to achieve the desired product properties.

The filtration may be typically carried out for a period of time from about 30 minutes to about 10 hours, or more typically from about 3 hours to about 7 hours. The filtration time may be determined by the desired moisture content of the filter cake. The moisture content may typically be in the range of from about 5% to about 50% residual moisture, or preferably from about 15% to about 40%. The filtration may typically be carried out at a temperature in the range of from about 0° C. to about 100° C., or preferably from about 20° C. to about 75° C.

The washing may be carried out for any amount of time needed to provide a regenerator iron oxide with a sufficiently reduced chloride content. The washing time may be divided among more than one washing cycle. The conductivity of the wash water may be measured to provide details on the effectiveness of the washing. For example, the regenerator iron oxide may be washed until the conductivity of the wash water is below a certain level, for example, below 100 μS, below 50 μS, or below 25 μS. A typical washing time may be from about 20 minutes to 20 hours, or preferably from about 1 hour to 10 hours. The washing may typically be carried out at a temperature from about 0° C. to about 100° C., or preferably from 20° C. to about 75° C.

The drying step may typically be carried out at a temperature ranging from 25° C. to about 350° C., or preferably from about 100° C. to about 200° C. The desired moisture content of the treated regenerator iron oxide may determine the drying time. The desired moisture content may be in the range of from about 0.1% to about 20% residual moisture, preferably from about 0.2% to about 5%, more preferably from about 0.2% to about 1%, and most preferably from about 0.2% to about 0.5%.

The type of filtration and drying equipment used may be any known to those skilled in the art. The filtration equipment may include a membrane filter press, a chamber filter press or a tube press. The drying equipment may be as simple as blowing hot air, or may include the use of a drying oven or such methods as spin flash drying.

The treated regenerator iron oxide may be dried, but the treated regenerator iron oxide is not calcined. The temperature in the treating, filtering, washing and drying steps should not exceed 350° C. Preferably, the temperature in these steps should be at most 300° C., or more preferably at most 200° C.

The chloride content of the treated regenerator iron oxide is at most 500 ppmw, relative to the weight of iron oxide, calculated as Fe₂O₃. The chloride content is preferably at most 300 ppmw, or more preferably at most 100 ppmw. The chloride content of the treated regenerator iron oxide is in the range of from 1 ppb to 500 ppmw, preferably from 1 ppmw to 300 ppmw, more preferably from 5 ppmw to 250 ppmw, and most preferably from 10 ppmw to 200 ppmw.

The treated regenerator iron oxide may have a BET surface area in the range of from about 2 m²/g to about 10 m²/g, or from about 3 m²/g to about 7 m²/g. The surface area of the treated regenerator iron oxide may be within a range of from about 50% of the surface area of the untreated regenerator iron oxide to about 200% of the surface area of the untreated regenerator iron oxide, or from about 75% to about 150%, or from about 90% to about 125%. The surface area may be at least 2 m²/g, at least 2.5 m²/g, at least 3 m²/g, or at least 3.5 m²/g. As the term is used herein, surface area is understood to refer to the surface area as determined by the BET (Brunauer, Emmett and Teller) method as described in the Journal of the American Chemical Society 60 (1938) pp. 309-316.

To prepare a catalyst, a mixture is prepared that comprises the treated regenerator iron oxide, a Column 1 metal or compound thereof and at least one additional catalyst component. The mixture may be prepared using any method known to those skilled in the art. The proportions of the catalyst components that are mixed together to form the mixture are such as to ultimately provide a dehydrogenation catalyst having an iron oxide content in the range of from 10 to 95 weight percent, based on the total weight of the final catalyst, and is calculated as Fe₂O₃. It is preferred for the iron oxide content of the catalyst to be in the range of from 40 to 90 weight percent, and more preferably in the range of from 60 to 85 weight percent.

In a preferred embodiment, the treated regenerator iron oxide is present in the mixture in an amount in the range of from 20 to 100 weight percent of the iron oxide, more preferably in the range of from 50 to 95 weight percent, and most preferably, from 75 to 92 weight percent, calculated as Fe₂O₃.

The mixture may also comprise yellow iron oxide. Yellow iron oxide is a hydrated iron oxide that is frequently depicted as α-FeOOH or Fe₂O₃.H₂O. The chloride content of the yellow iron oxide is preferably less than the chloride content of the treated regenerator iron oxide. Because sources of yellow iron oxide are generally those other than iron oxides produced by heat decomposition of iron chloride, the yellow iron oxide typically will have a chloride content less than that of the treated regenerator iron oxide. The chloride content of the yellow iron oxide is typically at most 500 ppmw, based on the weight of the yellow iron oxide. The chloride content of the yellow iron oxide is preferably at most 100 ppmw, and more preferably at most 50 ppmw.

The amount of yellow iron oxide used in the preparation of the catalyst mixture may be up to 50 weight percent of the total weight of the iron oxide. While it may be desirable to not use yellow iron oxide in the mixture, when yellow iron oxide is used, it is preferred for the amount of yellow iron oxide to be present in the mixture in an amount in the range of from 1 to 50 weight percent. More preferably, the amount of yellow iron oxide in the catalyst mixture is in the range of from 5 to 30 weight percent, and most preferably from 8 to 25 weight percent.

Minor amounts of other iron oxides or iron oxide providing compounds may be combined with the mixture, but the use of such other iron oxides or iron oxide providing compounds is generally not preferred or even desired. Examples of such other iron oxides include black iron oxide and red iron oxide. Examples of iron oxide providing compounds include goethite, hematite, magnetite, maghemite, lepidocricite and mixtures of any two or more thereof.

A Column 1 metal is generally added to the regenerator iron oxide as part of the catalyst preparation. A Column 1 metal is any metal from Column 1 of the Periodic Table, and the preferred Column 1 metal is potassium. Potassium is generally added as a potassium compound, for example potassium carbonate, potassium hydroxide, potassium oxide, or potassium oxalate. The amount of potassium compound in the mixture should be such as to provide a final catalyst having a potassium content in the range of from 5 to 40 weight percent, calculated as K₂O based on the total weight of the final catalyst. The potassium content may preferably be in the range of from 5 to 35 weight percent, or more preferably from 10 to 30 weight percent calculated as K₂O.

In addition to a Column 1 metal, additional catalyst components are generally added that act as promoters, stabilizers or otherwise provide beneficial qualities to the catalyst. Some typical additional catalyst components include Column 2 metals, cerium, molybdenum, and tungsten.

Column 2 metals, for example magnesium or calcium, or a combination thereof may be added to the regenerator iron oxide. The Column 2 metal component may be present in the final catalyst in an amount in the range of from 0.1 to 15 weight percent, calculated as the metal. The Column 2 metal content may preferably be in the range from 0.2 to 10 weight percent or more preferably from 0.3 to 5 weight percent, calculated as the metal.

Cerium may be added in an amount such as to provide a final catalyst having cerium content in the range of from 1 to 25 weight percent, calculated as cerium. The cerium content may preferably be in the range of from 2 to 20 weight percent, or more preferably from 3 to 15 weight percent, calculated as cerium.

Molybdenum, tungsten or a combination thereof may be added in an amount such as to provide a final catalyst having a molybdenum or tungsten content, or a combination thereof, of from 0.1 to 15 weight percent, calculated as the metal. The molybdenum, tungsten, or both metals may preferably be present in the final catalyst in an amount in the range of from 0.2 to 10 weight percent, or more preferably from 0.3 to 5 weight percent calculated as the metal.

Other additional catalyst components that may be incorporated in the mixture include scandium, yttrium, lanthanum, rubidium, vanadium, chromium, cobalt, nickel, manganese, copper, zinc, cadmium, aluminum, tin, bismuth, rare earths, and mixtures of any two or more thereof. Among these components, preferred are those selected from the group consisting of lanthanum, copper, vanadium, chromium, and mixtures of any two or more thereof.

The mixture may be formed or shaped into particles. The mixture may be formed or shaped into any form known to those skilled in the art as being a suitable type or shape for use as a catalyst particle. Examples of such shapes include extrudates, pellets, tablets, spheres, pills, saddles, trilobes, and tetralobes. One preferred method of preparing the particles is to mix together the catalyst components with water, or a plasticizer, or both, and form an extrudable paste from which extrudates are formed. The extrudates may be dried.

The mixture is subsequently calcined to yield a dehydrogenation catalyst. It is noted that in the process for preparing a dehydrogenation catalyst beginning with the starting regenerator iron oxide, throughout its treatment and up to the mixing of the treated regenerator iron oxide with at least one additional catalyst component, none of the steps include a calcination heat treatment of the materials. It is not until the mixture with additional catalyst component(s) is formed that a calcination step is utilized. The omission of a calcination step until this point in the process is a feature of the invention.

The mixture is calcined, preferably in an oxidizing atmosphere, such as air, at a temperature in the range from about 500° C. to about 1200° C., preferably from about 600° C. to about 1100° C., and more preferably from about 700° C. to about 1050° C. to form a catalyst.

The pore volume of the catalyst described herein is at least 0.01 ml/g, and more suitably, at least 0.05 ml/g. Concerning its upper limit, the pore volume is less than 0.5 ml/g, preferably less than 0.25 ml/g, and more preferably less than 0.20 ml/g.

The median pore diameter of the catalyst is at least 500 Å, and more suitably, at least 1000 Å. Concerning the upper limit, the median pore diameter of the catalyst is less than 10,000 Å, and preferably less than 8,000 Å. A most preferred catalyst has a median pore diameter in the range of from 1200 Å to 6500 Å.

As the term is used herein, the pore volume is as measured by mercury intrusion according to ASTM D4282-92, to an absolute pressure of 6000 psia (4.2×10⁷ Pa), using Micrometrics Autopore 9420 model (130° contact angle, mercury with a surface tension of 0.473 N/m). The term median pore diameter means the pore diameter at which 50% of the mercury intrusion volume is reached.

The surface area of the catalyst is in the range of from 0.01 to 20 m²/g. Preferably, the surface area is in the range of from 0.1 to 10 m²/g. The crush strength of the catalyst is suitably at least 10 N/mm, and more suitably it is in the range of from 20 to 100N/mm.

The catalyst comprising treated regenerator iron oxide described herein may suitably be used in the dehydrogenation of hydrocarbons. In the dehydrogenation method, the catalyst is contacted with a dehydrogenation feed under dehydrogenation reaction conditions to thereby provide a dehydrogenation reaction product. More specifically, the dehydrogenation feed is introduced into the dehydrogenation reactor wherein it is contacted with the dehydrogenation catalyst.

It is recognized that the dehydrogenation reactor or dehydrogenation reactor system can include more than one dehydrogenation reactor or reaction zone. If more than a single dehydrogenation reactor is used, they may be operated in series or in parallel, or they may be operated independently from each other and under the same or different process conditions.

The dehydrogenation feed can be any suitable feed and, more particularly, it can include any hydrocarbon that is dehydrogenatable. Examples of dehydrogenatable hydrocarbons include alkyl aromatics, such as alkyl substituted benzene and alkyl substituted naphthalene, isoamylenes, which can be dehydrogenated to isoprenes, and butenes, which can be dehydrogenated to butadiene. The preferred dehydrogenation feed comprises an alkylaromatic compound preferably one selected from the group of compounds consisting of ethylbenzene, propylbenzene, butylbenzene, hexylbenzene, methylpropylbenzene, methylethylbenzene, and diethylbenzene.

The most preferred dehydrogenation feed is an ethylbenzene feedstock comprising predominantly ethylbenzene. Ethylbenzene is dehydrogenated to styrene. The dehydrogenation feed can also include other components including diluents. It is common to use steam as a feed diluent when ethylbenzene is a feed component to be dehydrogenated to form styrene.

The dehydrogenation conditions can include a dehydrogenation reactor inlet temperature in the range of from about 500° C. to about 1000° C., preferably, from 525° C. to 750° C., and, most preferably, from 550° C. to 700° C. It is recognized that in the dehydrogenation of ethylbenzene to styrene the reaction is endothermic. When such a dehydrogenation reaction is carried out, it can be done so either isothermally or adiabatically. In the case where the dehydrogenation reaction is carried out adiabatically, the temperature across the dehydrogenation catalyst bed, between the dehydrogenation reactor inlet and the dehydrogenation reactor outlet, can decrease by as much as 150° C., but, more typically, the temperature can decrease from 10° C. to 120° C.

The reaction pressure is relatively low and can range from vacuum pressure upwardly to about 300 kPa. The absolute pressure is typically in the range of from 10 to 300 kPa, more typically from 20 to 200 kPa, for example 50 kPa, or 120 kPa. Due to the reaction kinetics of the dehydrogenation reaction of ethylbenzene to styrene it is generally preferable for the reaction pressure to be as low as is commercially feasible.

The liquid hourly space velocity (LHSV) can be in the range of from about 0.01 hr⁻¹ to about 10 hr⁻¹, and preferably, from 0.1 hr⁻¹ to 2 hr⁻¹. As used herein, the term “liquid hourly space velocity” is defined as the liquid volumetric flow rate of the dehydrogenation feed, for example, ethylbenzene, measured at normal conditions (i.e., 0° C. and 1 bar absolute), divided by the volume of the catalyst bed, or the total volume of catalyst beds if there are two or more catalyst beds.

When styrene is being manufactured by the dehydrogenation of ethylbenzene, it is generally desirable to use steam as a diluent usually in a molar ratio of steam to ethylbenzene in the range of from 0.1 to 20. Typically, the molar ratio of steam to ethylbenzene is in the range of from 2 to 15, and, more typically, from 4 to 12. Steam may also be used as a diluent with other dehydrogenatable hydrocarbons.

It is preferred for the dehydrogenation process conditions to be such as to provide for the conversion of the dehydrogenatable hydrocarbon compounds of the dehydrogenation feed to be in the range of from 30 to 100 mole percent, more preferably, at least 35 mole percent, and, most preferably, at least 40 mole percent. As used herein, the term “conversion” means the fraction, in mole %, of a specified compound converted to another compound. For ethylbenzene, conversion is defined as the difference between the moles of ethylbenzene in a dehydrogenation reactor feed and the moles of ethylbenzene in the dehydrogenation reactor effluent with the difference being multiplied by one hundred and divided by the moles of ethylbenzene in the dehydrogenation reactor feed.

The references herein to the activity of a catalyst are meant to refer to the temperature parameter associated with the particular catalyst. In the case of a styrene manufacturing catalyst, i.e., an ethylbenzene dehydrogenation catalyst, its temperature parameter is the temperature, in ° C., at which the styrene manufacturing catalyst provides under certain defined process conditions a specified conversion of an ethylbenzene feed. An illustrative example of activity is the temperature at which a conversion of 70 mole % of the ethylbenzene is achieved when contacted with the styrene manufacturing catalyst under certain reaction conditions. Such a temperature condition may be represented by the symbol “T₇₀”, which means that the given temperature provides for a conversion of 70 mole percent of the ethylbenzene in the feed. The T₇₀ temperature value represents the activity of the associated catalyst. The activity of a catalyst is inversely related to the temperature parameter with higher activities being represented by lower temperature parameters and lower activities being represented by higher temperature parameters.

The reference to initial activity is the activity of the fresh catalyst when it is first placed in use. For instance, the initial activity of a styrene manufacturing catalyst may be represented by its T₇₀ value at essentially the starting time at which it is first placed in use.

As used herein, the term “selectivity” means the fraction, in mole %, of the converted compound that yields the desired compound. As an example, in an ethylbenzene dehydrogenation process, the ethylbenzene of the feedstock is considered to be the converted compound and the desired compound is considered to be styrene. Thus, the selectivity is the mole % of the converted ethylbenzene that is converted into styrene.

Due to the unique catalytic properties of the treated regenerator iron oxide based dehydrogenation catalyst, such as its high initial activity, the operation of conventional dehydrogenation process systems that include a dehydrogenation reactor loaded with either a non-regenerator iron oxide based dehydrogenation catalyst or a conventional regenerator iron oxide based dehydrogenation catalyst can be improved by removing from the dehydrogenation reactor the catalyst load and replacing it with a treated regenerator iron oxide based dehydrogenation catalyst as is described in detail herein. The thus-loaded or reloaded dehydrogenation reactor is a modified dehydrogenation system including a dehydrogenation reactor loaded with a treated regenerator iron oxide based dehydrogenation catalyst having the low chloride concentration as noted herein.

As it is referred to in this specification, the conventional regenerator iron oxide based dehydrogenation catalyst is a catalyst composition that is based on an iron oxide component in that its iron oxide component comprises predominantly regenerator iron oxide having a chloride content greater than 500 ppmw.

The dehydrogenation system that includes a reactor loaded with a treated regenerator iron oxide based dehydrogenation catalyst is operated by contacting a dehydrogenation feed under dehydrogenation reaction conditions to thereby provide a dehydrogenation reaction product. More specifically, the dehydrogenation feed is introduced into the dehydrogenation reactor containing the treated regenerator iron oxide based dehydrogenation catalyst wherein it is contacted with the catalyst.

The following Examples are presented to illustrate the invention, but they should not be construed as limiting the scope of the invention.

### EXAMPLE I

This Example presents information on the properties of the regenerator iron oxides A, B, and C used in the preparation of Catalysts 1, 2 and 3 and descriptions on how the Catalysts were prepared.

Table I presents certain of the chemical and physical properties of iron oxides A, B and C. The iron oxides A and B are typical iron oxides prepared by the heat decomposition of iron chloride (regenerator iron oxide) and which were not subjected to additional treating, calcination or other chloride reduction treatments. Iron oxide C was obtained from the same supplier of the iron oxides A and B but was further treated by water washing in accordance with the invention to further reduce the chloride content.

TABLE 1
Selected Chemical and Physical Properties of
Iron Oxides A, B, and C

| | | | Untreated | Treated |
|---|---|---|---|---|
| | | | Iron | Iron |
| Property | Iron Oxide A | Iron Oxide B | Oxide C | Oxide C |
| Cl (wt. %) | 0.098 | 0.111 | 0.088 | 0.029 |
| SiO₂ (wt. %) | 0.009 | 0.010 | 0.008 | 0.011 |
| Na₂O (wt. %) | 0.010 | 0.011 | 0.007 | 0.003 |
| CaO (wt. %) | 0.013 | 0.015 | 0.013 | 0.004 |
| MnO (wt. %) | 0.264 | 0.269 | 0.266 | 0.258 |
| Cr (wt. %) | 0.018 | 0.016 | 0.015 | 0.016 |
| Cu (wt. %) | 0.010 | 0.010 | 0.006 | 0.006 |
| Al₂O₃ (wt. %) | 0.058 | 0.065 | 0.0065 | 0.066 |
| P (wt. %) | 0.009 | 0.010 | 0.009 | 0.009 |
| MgO (wt. %) | 0.007 | 0.009 | 0.006 | 0.003 |
| TiO₂ (wt. %) | 0.009 | 0.012 | 0.007 | 0.010 |
| Ni (wt. %) | 0.017 | 0.021 | 0.015 | 0.014 |
| Apparent | 0.58 | 0.57 | 0.52 | 0.59 |
| density | | | | |
| BET | 3.47 | 3.51 | 3.41 | 3.76 |

As can be seen from the table, the water washing removes other impurities in addition to chlorides. Removing these impurities is an additional advantage of this invention. Also, the table demonstrates that the treated iron oxide does not have a smaller surface area than the untreated iron oxide. In fact, in this example, the treated iron oxide has a larger surface area than the untreated iron oxide.

Catalyst 1 was prepared by first forming a paste by mixing untreated iron oxide A, yellow iron oxide (Bayer, type 920Z), potassium carbonate, cerium carbonate, molybdenum trioxide, calcium carbonate, and water (about 9 wt. %, relative to the weight of the dry mixture). The paste was extruded to form 3 mm diameter cylinders cut into 6 mm length extrudate particles. The pellets were dried in air at 170° C. for 15 minutes and subsequently calcined in air at 825° C. for 1 hour. After calcination, the composition of the catalyst was, on a per mole of iron oxide (as Fe₂O₃) basis, 0.516 mole potassium, 0.066 mole cerium, 0.022 mole molybdenum, and 0.027 mole calcium. The quantity of yellow iron oxide was sufficient to provide 8.8 mole %, as Fe₂O₃, relative to the total moles of iron oxide, as Fe₂O₃, in the catalyst.

Catalyst 2 was prepared in the same manner as described above for Catalyst 1; except that, the untreated iron oxide B was used in place of untreated iron oxide A.

Catalyst 3 was prepared in the same manner as described above for Catalyst 1; except that, treated iron oxide C was used in place of untreated iron oxide A.

### EXAMPLE II

This Example describes the procedure by which the Catalysts 1, 2 and 3 of Example I were tested, and it presents the results of such testing.

A 100 ml sample of each of the Catalysts described in Example I was used for the preparation of styrene from ethylbenzene under isothermal testing conditions in a reactor designed for continuous operation. In each test, the conditions were as follows: absolute pressure of 76 kPa, steam-to-ethylbenzene molar ratio of 10, liquid hourly space velocity of 0.65 liter/liter/hr. In each test, the reactor temperature was maintained at 600° C. for a period of 7 to 10 days. The reactor temperature was thereafter adjusted until a 70% mole conversion of the ethylbenzene was achieved.

Presented in FIG. 1 and FIG. 2 are results of the above-described testing. FIG. 1 shows the calculated catalyst activity (T₇₀) plotted as a function of time (days) in use. FIG. 2 shows the actual ethylbenzene conversion plotted as a function of time (days). Catalyst 3 achieves a high conversion shortly after startup, but Catalyst 1 and Catalyst 2 require more than one week to achieve stable performance at the target conversion. As may further be observed from the plots, Catalysts 1 and 2 exhibit significantly lower initial activities than Catalyst 3. Also, Catalyst 3 exhibited a relatively stable activity in that the activity of the catalyst did not significantly change from its initial activity with time in use. However, Catalysts 1 and 2 demonstrated an increasing activity with time in use from their initial activity. The rate of increase in the activity of the Catalysts 1 and 2 tended to decline during an initial break-in period to a point in time at which their activity stabilized at an activity level comparable to that of Catalyst 3. Due to the higher initial and relatively stable activity of Catalyst 3, its use in a dehydrogenation process can provide significant operational and economic benefits by eliminating the need for a break-in period. Catalysts 1, 2 and 3 respectively exhibited selectivities, S₇₀, of 95.5%, 95.1% and 96.0%.

Reasonable variations, modifications and adaptations of the invention may be made within the scope of the described disclosure and appended claims without departing from the spirit and scope of the invention.

What is claimed:

1. A process for preparing a dehydrogenation catalyst comprising preparing a mixture comprising a treated regenerator iron oxide and at least one additional catalyst component; and calcining the mixture wherein the treated regenerator iron oxide is prepared by washing a regenerator iron oxide at a temperature below 350° C. such that the treated regenerator iron oxide has a chloride content of at most 500 ppmw relative to the weight of iron oxide, calculated as Fe₂O₃.

2. A process as claimed in claim 1 wherein the regenerator iron oxide prior to washing has a chloride content of at least 700 ppmw relative to the weight of iron oxide, calculated as Fe₂O₃.

3. A process as claimed in claim 1 or claim 2 wherein the treated regenerator iron oxide has a chloride content of at most 300 ppmw relative to the weight of iron oxide, calculated as Fe₂O₃.

4. A process as claimed in any of claims 1-3 wherein the treated regenerator iron oxide is prepared by washing the regenerator iron oxide with water.

5. A process as claimed in any of claims 1-4 wherein the treated regenerator iron oxide is prepared by washing the regenerator iron oxide with an acidic solution.

5. A process as claimed in claim 5 wherein the acidic solution is selected from the group consisting of acetic acid, formic acid and citric acid.

7. A process as claimed in claim 4 wherein the process comprises contacting the regenerator iron oxide with water to form a slurry, contacting the slurry with an aqueous solution having a pH higher than the pH of the slurry, drying the slurry and washing the dried slurry with water.

8. A process as claimed in claim 7 wherein the slurry is heated to a temperature of at most 200° C.

9. A process as claimed in any of claims 1-8 wherein the additional catalyst component comprises a metal selected from the group consisting of calcium, magnesium, molybdenum, tungsten, cerium, and combinations thereof.

10. A process as claimed in any of claims 1-8 wherein potassium or a compound thereof is added to the treated regenerator iron oxide mixture.

11. A process as claimed in any of claims 1-10 wherein the mixture is calcined at a temperature of from about 600° C. to about 1300° C.

12. A catalyst produced by the process of any of claims 1-11 wherein the dehydrogenation catalyst comprises from 10 to 95 weight percent iron oxide relative to the total weight of the dehydrogenation catalyst, calculated as Fe₂O₃, and from 5 to 40 weight percent potassium, calculated as K₂O.

13. A catalyst as claimed in claim 12 wherein the dehydrogenation catalyst further comprises from 1 to 25 weight percent cerium, calculated as CeO₂.

14. A dehydrogenation process, comprising: providing a reactor loaded with a catalyst as claimed in claim 12 or claim 13, introducing a feedstock into the reactor, and operating the reactor under conditions suitable to yield a dehydrogenation product.

15. A method of improving the operation of a dehydrogenation system, including a dehydrogenation reactor that is loaded with a non-regenerator iron oxide based dehydrogenation catalyst, wherein said method comprises: removing from said dehydrogenation reactor said non-regenerator iron oxide based dehydrogenation catalyst; replacing the thus-removed non-regenerator iron oxide based dehydrogenation catalyst with a regenerator iron oxide based dehydrogenation catalyst as claimed in claim 12 or claim 13 to thereby provide a modified dehydrogenation system; and operating said modified dehydrogenation system under dehydrogenation process conditions.
